(19) United States
(12) Patent Application Publication
(10) Pub. No.: US 2024/0226030 A1
(43) Pub. Date: Jul. 11, 2024

(54) COMPOSITIONS AND METHODS USING AT LEAST ONE OF INOSITOL, ERYTHRITOL OR SORBITOL TO ENHANCE GROWTH OF FAECALIBACTERIUM PRAUSNITZII

(71) Applicant: SOCIÉTÉ DES PRODUITS NESTLE S.A., Vevey (CH)

(72) Inventors: CHIEH JASON CHOU, Chexbres (CH); SHAILLAY KUMAR DOGRA, Epalinges (CH); ADRIEN DARDINIER, Lausanne (CH)

(21) Appl. No.: 18/558,956

(22) PCT Filed: May 4, 2022
(86) PCT No.: PCT/EP2022/061949

§ 371 Date: Nov. 3, 2023

Related U.S. Application Data
(60) Provisional application No. 63/185,097, filed on May 6, 2021.

(51) Int. Cl. A61K 31/047; A61K 31/07; A61K 31/197; A61K 31/352; A61K 31/366; A61K 31/4415; A61K 31/593; A61K 31/714

(52) U.S. Cl. CPC A61K 31/047; A61K 31/07; A61K 31/197; A61K 31/352; A61K 31/366; A61K 31/4415; A61K 31/593; A61K 31/714

(57) ABSTRACT

An aspect is a composition containing at least one of inositol, erythritol or sorbitol and formulated for administration to a subject such that the at least one of inositol, erythritol or sorbitol enhance growth of Faecalibacterium prausnitzii in the microbiome of the subject. Another aspect is a method to treat, prevent, reduce a severity of, and/or reduce an incidence of a pathological condition in the subject, and the method includes enhancing growth of Faecalibacterium prausnitzii in the microbiome of the subject by administering, to the subject, at least one of inositol, erythritol or sorbitol. Yet another aspect is a method to achieve at least one of a metabolic effect, an anti-aging effect, or an anti-inflammatory effect in a subject, and the method includes enhancing growth of Faecalibacterium prausnitzii in the microbiome of the subject by administering, to the subject, at least one of inositol, erythritol or sorbitol.

### BACKGROUND

The present disclosure generally relates to compositions comprising at least one of inositol, erythritol or sorbitol in an amount effective to enhance growth of Faecalibacterium prausnitzii. The present disclosure further relates to methods of making such compositions. The present disclosure also relates to methods of using such compositions therapeutically or prophylactically, for example for health benefits related to increased Faecalibacterium prausnitzii status.

Increasing scientific evidence indicates the importance of gut microbiota in human health. Although it is well known diet plays a critical role in shaping the function and composition of the gut microbiota, the optimal nutritional composition to maximize the benefits of gut microbiota remains unknown.

### SUMMARY

Butyrate producers are members of beneficial bacteria in microbiota. For example, butyrate-producing bacteria play a key role in colonic health of humans. Despite sharing a common ability to produce butyrate, members sharing this function are diverse in phylogenic groups and rely on different metabolic pathways to generate butyrate. Major butyrate producing bacteria isolated from human colon are Gram-positive anaerobic bacteria, and many of them belong to the phylum Firmicutes and the cluster of Clostridial IV, XIVa and XVI [1]. Two of the most important groups in human guts are Faecalibacterium prausnitzii (F. prausnitzii) and Eubacterium rectale/Roseburia spp.), and some species in Coprococcus genus (C. catus, C. eutactus and C. comes) are also known to produce butyrate.

Butyrate is one of major short chain fatty acids results from the fermentation of undigestable carbohydrate by butyrate producers. In humans, butyrate is essential to maintain the homeostasis of colonic environment by serving as a major energy source to colonic epithelial cells and being a key regulator the immune system [2,3]. In addition to producing butyrate, the abundance of the butyrate producers also coincides with many human conditions. In gut-related diseases, a low number of F. prausnitzii was found in Crohn's disease [4]. Similarly, Roseburia was lower in colorectal cancer [5] and constipation-predominant IBS [6]. Reduction of F. prausnitzii was also reported in patients with type 2 diabetes and metabolic syndrome [7,8]. Conversely, people with improved insulin sensitivity had a community high in the abundance of butyrate producers [9]. The mechanisms involved in butyrate's ability to modulate immune system and regulation of mitochondrial functions in host cells have been shown to influence the intestinal homeostasis and other organs beyond the digestive tract. In the gut, butyrate producers are a functionally unique group of bacteria and, its abundance is a major indicator of disease conditions in humans.

F. prausnitzii is a butyrate-producing bacteria, but the benefits of F. prausnitzii are beyond butyrate production. For example, F. prausnitzii is an important bacteria in the human gut microbiome ecosystem with an associative to causative role in different conditions such as its importance in human health [10,11], anti-inflammatory [12,13], ulcerative colitis [14], Crohn's disease [15], child allergy such as asthma [16]; IBD [17,18] and frailty [19].

Additionally, F. prausnitzii is affected in multiple stressful conditions to the gut microbiome ecosystem such as a drastic diet change or antibiotics usage. For example, Mardinoglu et al. (Cell Metabolism 2018) showed a decrease in F. prausnitzii under a ketogenic diet challenge. Similarly, Palleja et al. (Nature Microbiology 2018) showed a decrease in F. prausnitzii under an antibiotics challenge. Furthermore, David et al. (Nature 2014) provided evidence of a decrease in F. prausnitzii abundance under high fat diet challenge.

As set forth in the experimental examples disclosed later herein, a study was conducted to discover nutrient features differentiating F. prausnitzii abundance. A model organism confirmed the effect of inositol, erythritol and sorbitol as an energy substrate for F. prausnitzii. Furthermore, a combination of coumestrol with sorbitol or inositol enhanced growth of F. prausnitzii more than each ingredient alone, and addition of quercetin to sorbitol enhanced growth for the strain A2-165 of F. prausnitzii.

Accordingly, embodiments provided by the present disclosure include a composition comprising (i) at least one of inositol, erythritol or sorbitol, (ii) optionally a polyphenol such as coumestrol and/or quercetin, and (iii) optionally a vitamin such as one or more of Vitamin B5, Vitamin B6, Vitamin B12, Vitamin A or Vitamin D. In some embodiments, a unit dosage form comprises an amount of the composition effective to enhance growth of F. prausnitzii in a subject to whom the composition is administered, preferably orally. In some embodiments, the composition is administered to a subject to treat, prevent, reduce a severity of, and/or reduce an incidence of a pathological condition, for example by exerting one or more metabolic, anti-aging or anti-inflammatory effects from enhanced growth of F. prausnitzii.

Additional features and advantages are described herein and will be apparent from the following Figures and Detailed Description.

### BRIEF DESCRIPTION OF DRAWINGS

FIGS. 1A and 1B are respectively the Train ROC curve and the holdout/Test curve from the experimental examples disclosed herein, showing model performance based on nutrient features only.

FIGS. 2A and 2B depict the top thirty (30) nutrient features in the model from the experimental examples disclosed herein.

FIGS. 3A-3D show results from sugar alcohols in the experimental examples disclosed herein.

FIGS. 4A and 4B show results from the experimental examples disclosed herein, confirming the effect of inositol and sorbitol as energy substrate for F. prausnitzii.

FIGS. 5A-5L show results for the polyphenols coumestrol and quercetin from the experimental examples disclosed herein.

FIGS. 6A-6D show results for B Vitamins from the experimental examples disclosed herein.

FIGS. 7A and 7B show results for Vitamin A from the experimental examples disclosed herein.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about”, “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a vitamin” or “the vitamin” encompass both an embodiment having a single vitamin and an embodiment having two or more vitamins.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The terms “at least one of” and “and/or” used in the respective context of “at least one of X or Y” and “X and/or Y” should be interpreted as “X,” or “Y,” or “X and Y.” For example, “at least one of inositol or sorbitol” and “inositol and/or sorbitol” should be interpreted as “inositol without sorbitol,” or “sorbitol without inositol,” or “both inositol and sorbitol.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual. The relative terms “promote,” “improve,” “increase,” “enhance” and the like refer to an enhanced status of F. prausnitzii in the microbiome of the subject, after administration of the composition disclosed herein (which comprises sorbitol, erythritol and/or inositol), relative to the status of F. prausnitzii in the microbiome of the subject obtained by administration of a composition lacking sorbitol, erythritol and inositol but otherwise identically formulated. As used herein, “enhanced growth” of F. prausnitzii is used interchangeably with an “enhanced status” of F. prausnitzii.

This enhanced status of F. prausnitzii in the microbiome of the subject can be characterized by at least one or more of (i) a higher total amount of F. prausnitzii in the microbiome of the subject (i.e., total cfu of Faecalibacterium prausnitzii) or (ii) a higher relative percentage of Faecalibacterium prausnitzii compared to the other bacteria in the microbiome of the subject (i.e., cfu of Faecalibacterium prausnitzii/cfu of other bacteria).

As used herein, the terms “food,” “food product” and “food composition” mean a product or composition that is intended for oral ingestion by a human or other mammal and comprises at least one nutrient for the human or other mammal.

“Nutritional compositions” and “nutritional products,” as used herein, include any number of food ingredients and possibly optional additional ingredients based on a functional need in the product and in full compliance with all applicable regulations. The optional ingredients may include, but are not limited to, conventional food additives, for example one or more, acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount.

“Probiotic” means microbial cell preparations or components of microbial cells with a beneficial effect on the health or well-being of the host. (Salminen S, Ouwehand A. Benno Y. et al “Probiotics: how should they be defined” Trends Food Sci. Technol. 1999:10 107-10).

The term “metabolic conditions” as used herein refers to metabolic disorders that negatively alters the body's processing and distribution of macronutrients such as proteins, fats, and carbohydrates. Metabolic disorders can happen when abnormal chemical reactions in the body alter the normal metabolic process. This may include diabetes (type 1 and 2), Gaucher's disease, Glucose galactose malabsorption, hereditary hemochromatosis, Maple syrup urine disease, phenylketonuria, Hunter syndrome, etc. It also relates to metabolic syndrome which is associated with the risk of developing type 2 diabetes but also cardiovascular disease. Metabolic syndrome is a clustering of at least three of the following five medical conditions: abdominal obesity, high blood pressure, high blood sugar, high serum triglycerides, and low serum high-density lipoprotein (HDL).

The term “unit dosage form,” as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

A “subject” or “individual” is a mammal, preferably a human.

### EMBODIMENTS

An embodiment provided by the present disclosure is a composition comprising at least one of inositol, erythritol or sorbitol and formulated for administration to a subject such that the at least one of inositol, erythritol or sorbitol enhance growth of Faecalibacterium prausnitzii in the microbiome of the subject.

Another aspect is a method to treat, prevent, reduce a severity of, and/or reduce an incidence of a pathological condition in a subject, the method comprising enhancing growth of Faecalibacterium prausnitzii in the microbiome of the subject by administering, to the subject, at least one of inositol, erythritol or sorbitol.

Yet another aspect is a method to achieve at least one of a metabolic effect, an anti-aging effect, or an anti-inflammatory effect in a subject, the method comprising enhancing growth of Faecalibacterium prausnitzii in the microbiome of the subject by administering, to the subject, at least one of inositol, erythritol or sorbitol.

Another aspect is a unit dosage form of a composition comprising at least one of inositol, erythritol or sorbitol, the unit dosage form comprising an amount of the at least one of inositol, erythritol or sorbitol effective to enhance growth of Faecalibacterium prausnitzii in the microbiome of a subject to whom the unit dosage form is administered.

Yet another aspect is a method of making a composition to enhance growth of Faecalibacterium prausnitzii in the microbiome of a subject to whom the composition is administered, the method comprising adding at least one of inositol, erythritol or sorbitol to at least one additional component.

The inositol, erythritol and/or sorbitol is preferably orally administered in a composition such as a food composition. The inositol, erythritol and/or sorbitol can be the only sugar alcohol(s) in the composition or alternatively the composition can comprise one or more additional sugar alcohols.

In some embodiments, the inositol, erythritol and/or sorbitol are a total amount that is at least 50 wt. % of all sugar alcohols in the composition, preferably at least 60 wt. % of all sugar alcohols in the composition, more preferably at least 70 wt. % of all sugar alcohols in the composition, even more preferably at least 80 wt. % of all sugar alcohols in the composition, most preferably at least 90 wt. % of all sugar alcohols in the composition.

The inositol, erythritol and/or sorbitol is preferably administered in a composition further comprising a polyphenol. The polyphenol can comprise at least one of coumestrol or quercetin, for example as the only polyphenol(s) in the composition or alternatively in combination with one or more other polyphenols. In some embodiments, the at least one of coumestrol or quercetin are a total amount that is at least 50 wt. % of all polyphenols in the composition, preferably at least 60 wt. % of all polyphenols in the composition, more preferably at least 70 wt. % of all polyphenols in the composition, even more preferably at least 80 wt. % of all polyphenols in the composition, most preferably at least 90 wt. % of all polyphenols in the composition.

The inositol, erythritol and/or sorbitol is preferably administered in a composition further comprising a vitamin. The vitamin can comprise at least one of Vitamin B5, Vitamin B6, Vitamin B12, Vitamin A or Vitamin D, for example as the only vitamin(s) in the composition or alternatively in combination with one or more other vitamins. In some embodiments, the at least one of Vitamin B5, Vitamin B6, Vitamin B12, Vitamin A or Vitamin D are a total amount that is at least 50 wt. % of all vitamins in the composition, preferably at least 60 wt. % of all vitamins in the composition, more preferably at least 70 wt. % of all vitamins in the composition, even more preferably at least 80 wt. % of all vitamins in the composition, most preferably at least 90 wt. % of all vitamins in the composition.

Administration of the inositol, erythritol and/or sorbitol can treat, prevent, reduce a risk of, reduce a severity of, and/or reduce an incidence of a pathological condition in the subject. For example, the pathological condition can comprise at least one of a metabolic condition, an inflammatory condition, inflammatory bowel disease such as ulcerative colitis or Crohn's disease, child allergy such as asthma, multiple sclerosis, aging, or frailty. In some embodiments, the inositol, erythritol and/or sorbitol enhance growth of Faecalibacterium prausnitzii in the microbiome of the subject to thereby exert at least one of a metabolic effect, an anti-aging effect, or an anti-inflammatory effect in the subject.

The subject to whom the inositol, erythritol and/or sorbitol is administered can be selected from the group consisting of a human infant, a human child, a human adolescent, a human adult and an elderly human.

In some embodiments, the inositol, erythritol and/or sorbitol is administered in a composition further comprising one or more probiotics. Alternatively, the inositol, erythritol and/or sorbitol can be administered in a composition that does not contain any probiotic in the composition.

The inositol, erythritol and/or sorbitol can be administered to the individual by at least one route selected from the group consisting of oral, topical, enteral and parenteral. For example, the inositol, erythritol and/or sorbitol can be administered in a composition selected from the group consisting of a nutritionally complete product, a drink, a dietary supplement, a meal replacement, a food additive, a supplement to a food product, a powder for dissolution, an enteral nutrition product, an infant formula, and combinations thereof.

In some embodiments, the inositol, erythritol and/or sorbitol can be administered in an amount effective to treat, prevent, reduce an incidence of, and/or reduce a severity of inflammation. Non-limiting examples of such inflammation can be selected from the group consisting of acute inflammation, skin inflammation, inflammatory bowel disease (IBD) including Crohn's disease and/or ulcerative colitis, irritable bowel syndrome, liver inflammation (NASH, NAFLD, alcohol-induced liver injury), allergy, atopy, bone inflammation, rheumatoid arthritis, systemic lupus, Gougerot-Sjogren's syndrome, Reiter's syndrome, poliomyelitis, dermato-myositis, thyroiditis, Basedow, Hashimoto, type I diabetes, Addison's disease, auto-immunes hepatitis, celiac disease, Biermer's disease, multiple sclerosis, myasthenia, eye inflammation, obesity-associated inflammation, age-related low-grade inflammation, Blau's syndrome, Alzheimer's disease, cardiovascular diseases, atherosclerosis, metabolic syndrome, type II diabetes, gingivitis, paronditis, food sensitivities, Celiac disease, and combinations thereof.

The inflammation treated or prevented by the inositol, erythritol and/or sorbitol can be IBD, for example Crohn's disease or ulcerative colitis.

In some embodiments, the subject who is administered the inositol, erythritol and/or sorbitol is selected from the group consisting of an infant, a child, an adolescent, an adult and an elderly person.

Optionally the inositol, erythritol and/or sorbitol is administered in a composition further comprising at least one component selected from the group consisting of a prebiotic, an amino acid, a protein, a nucleotide, a fish oil, a non-marine source of omega-3 fatty acids, a phytonutrient, an antioxidant, and mixtures thereof.

“Prebiotic” means a food substance that promote the growth of beneficial bacteria in the intestines. A prebiotic is not broken down in the stomach or absorbed in the GI tract of the individual ingesting the prebiotic, but the prebiotic is fermented by the gastrointestinal microflora and/or by probiotics. The prebiotics that may be used with the at least one of inositol, erythritol or sorbitol are not particularly limited and include all food substances that promote the growth of probiotics in the intestine. Preferably, the prebiotic may be selected from the group consisting of oligosaccharides, optionally containing fructose, galactose, mannose; dietary fibers, in particular soluble fibers, soy fibers; inulin; or mixtures thereof. Preferred prebiotics are fructo-oligosaccharides (FOS), galacto-oligosaccharides (GOS), isomalto-oligosaccharides, xylo-oligosaccharides, oligosaccharides of soy, glycosylsucrose (GS), lactosucrose (LS), lactulose (LA), palatinose-oligosaccharides (PAO), malto-oligosaccharides, hemicellulosic polysaccharides (eg. Arabinoxylans), pectins and/or hydrolysates thereof.

The composition comprising the inositol, erythritol and/or sorbitol may be a food product, an animal food product, or a pharmaceutical composition. For example, the product may be a nutritional composition, a nutraceutical, a drink, a food additive or a medicament. A food additive or a medicament may be in the form of tablets, capsules, pastilles, a liquid, or a powder in a sachet, for example.

The composition comprising the inositol, erythritol and/or sorbitol is preferably selected from the group consisting of milk powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavoured drinks; culinary products; soups; tablets; and/or syrups.

The composition may optionally comprise any milk obtainable from animal or plant sources, such as one or more of cow's milk, human milk, sheep milk, goat milk, horse milk, camel milk, rice milk or soy milk. Additionally or alternatively, milk-derived protein fractions or colostrum may be used.

The composition comprising the inositol, erythritol and/or sorbitol may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition comprising the inositol, erythritol and/or sorbitol may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. Further, the composition comprising the inositol, erythritol and/or sorbitol may contain an organic or inorganic carrier material suitable for oral or enteral administration as well as vitamins, minerals trace elements and other micronutrients in accordance with the recommendations of Government bodies such as the USRDA.

The composition comprising the inositol, erythritol and/or sorbitol may optionally contain one or more amino acids, a protein source, a carbohydrate source and/or a lipid source, particularly in embodiments of the composition that are a food product.

Any suitable dietary protein may be used, for example animal proteins (such as milk proteins, meat proteins and egg proteins); vegetable proteins (such as soy protein, wheat protein, rice protein, and pea protein); mixtures of free amino acids; or combinations thereof. Milk proteins such as casein and whey, and soy proteins are particularly preferred.

The composition comprising the inositol, erythritol and/or sorbitol may be administered to humans or animals, in particular companion animals, pets or livestock. It has beneficial effects for any age group. Preferably, the composition is formulated for administration to infants, juveniles, adults or elderly. In some embodiments, the composition can be administered to mothers during pregnancy and lactation to treat the infant.

The composition comprising the inositol, erythritol and/or sorbitol can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

### EXAMPLES

The following non-limiting examples generally illustrate the concepts underlying the embodiments disclosed herein.

### Example 1: Converting Food Frequency Intake Data to Nutrients

Food intake data from the participants of a publicly available citizen-science project called the American Gut Project (AGP) (McDonald D., et al. mSystems. 2018) was converted into nutrients intake using a tool called vioscreen and provided to the public by AGP.

### Example 2: Building Models to Estimate Relative Amounts of Faecalibacterium prausnitzii (Fprau)

Predictive models were built to determine the relative amounts of Faecalibacterium prausnitzii (Fprau) of an individual subject. In particular, the model predicted the Fprau relative amounts by several feature parameters to determine whether a subject has “Low” or “notLow”; “High” or “notHigh”; “Low” or “High” Fprau amounts; as per the categories defined above.

A cube-root transformation was performed to make the amounts of Fprau normally distributed before binning them in different categories. The values for various definition of bins were: first/lower quartile—0.2819, third/upper quartile—0.4666, mean-std—0.1954, and mean+std—0.5220.

For building the classification models, the data was split into a training set “Train” and a testing set “holdout/Test set.” For optimal model performance, down-sampling balanced the imbalanced classes, which may occur based on the definition of bins.

The Train set was used by a machine learning algorithm to train the model. This involved finding variables (i.e., features) and thresholds (or coefficients) to use for classifying the groups. The learning from the data was done in a cross-validated manner where Train data was split into partitions with some parts used for training the model and other parts used for internal testing (k-Fold Cross-Validation, for ex., 3-folds), or with this process also repeated a few times (Repeated k-Fold Cross-Validation, for ex., 10-folds, 10-repeats).

The holdout/Test set was used only for checking the performance of the final trained model. This holdout/Test dataset was thus not used during the model training phase. The study evaluated multiple statistical models (different machine learning algorithms) using freely available tools (R software, python) and identified the best models for Low vs. notLow, High vs. notHigh, and Low vs. High for Fprau amounts.

Evaluating the model performance was critical, during all phases of modeling. Once the model was trained, it was applied on holdout/Test data, which was not used during the training phase. The model computed probabilities to be in each group (e.g. “Low”, “notLow”). A final decision was made based on this probability, which thus required the use of a threshold. This threshold impacted the final classification for a subject, whether a subject was correctly classified or not. Thus, the error was evaluated for different choices of threshold. For each given threshold, a confusion matrix was computed. This confusion matrix essentially listed the number of correctly and incorrectly classified subjects. By using different thresholds, one generated many confusion matrices, which were used to derived sensitivity and specificity at different thresholds. These two metrics—sensitivity and specificity—were commonly shown in the form of a Receiving Operating Curve (ROC); which summarized the model performance over several threshold values.

Receiver Operating Characteristic (ROC) curves were produced for the model. The study defined the group of “Low” subjects (and in “notLow” group) and predicted the probability of subject to be in this group; or defined the subjects to be in the “High” group (and in “notHigh” group) and predicted the probability of subject to be in this group; or defined the subjects to be in the “Low” group (and in “High” group) and predicted the probability of subject to be in this group.

As mentioned earlier, the data set used for the examples of a predictive model was from the American Gut Project (AGP) database (http://americangut.org).

### Example 3: Estimating “Low” Fprau Amounts from Nutrient Intake Data (I)

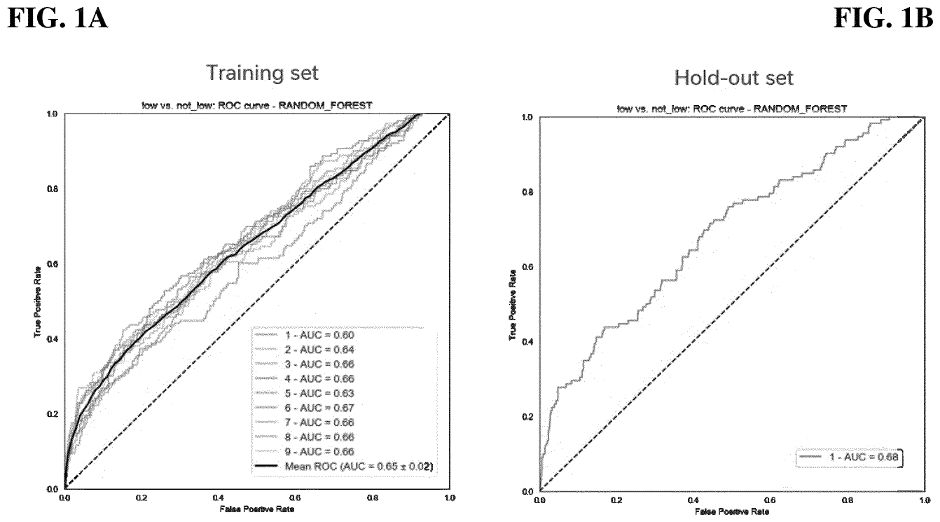

A model for Low vs. notLow Fprau amounts was learned with these parameters: Bin definition: (mean—1*std) vs rest; Features cut-offs: None; Algorithm: Random Forest; Train mode: cv-splits-3, cv-repeats-3; Post Process Train size: 896; and holdout/Test size (original/before preprocess train/test split): 764 (Test percentage: 20.0%). The results obtained for Train in cross-validation were: Accuracy—0.58+0.02, Sensitivity—0.61+0.05, and Specificity—0.58+0.03. The Train ROC curve is shown in FIG. 1A. The results obtained for holdout/Test set were: Accuracy—0.64, Sensitivity—0.56, and Specificity—0.65. The holdout/Test ROC curve is shown in FIG. 1B.

### Example 4: Nutrition to Maintain or Improve Fprau Amounts

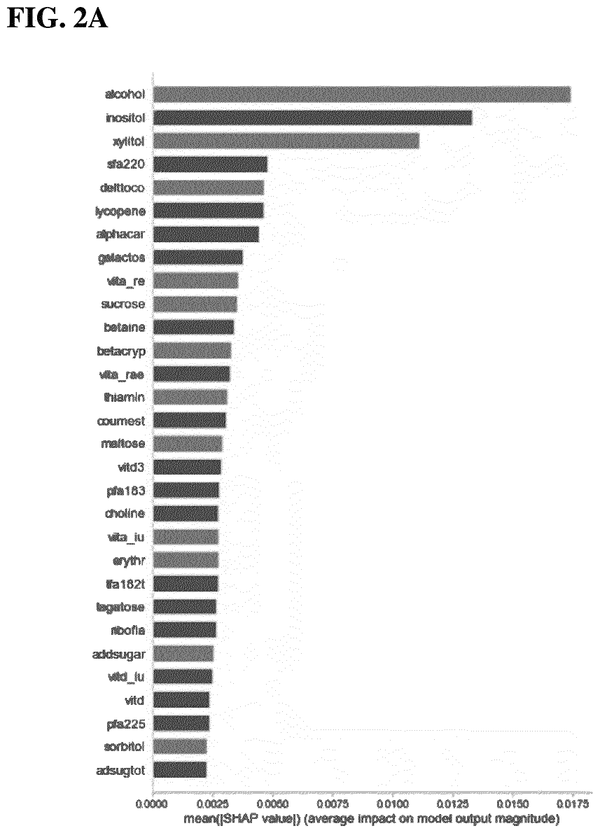

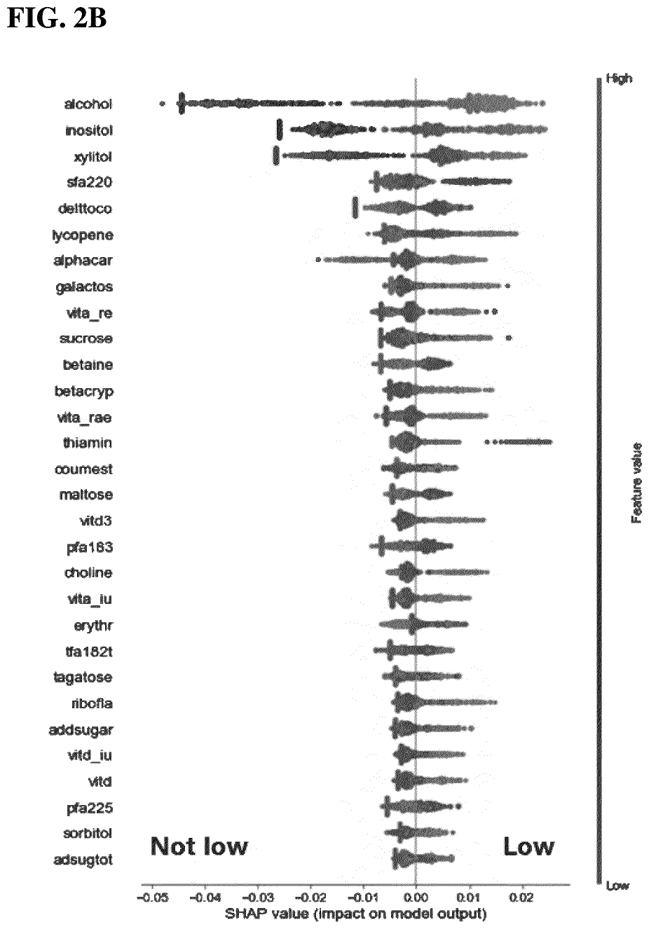

The important features and their association with Fprau amounts are shown in FIGS. 2A and 2B. Specifically, for the model presented in Example 3, the top thirty (30) features that constitute the model are shown in FIGS. 2A and 2B, which were obtained by running SHAP algorithm analyses. FIG. 2A shows the average impact per feature on the model output in their order of importance from high to low. The main/best feature is the top horizontal bar. The next best feature is the second horizontal bar, and so on. FIG. 2B shows in more detail the impact of a feature per instance/sample on the model output. The color gradation from grey to black indicates low to high values for that feature. The vertical line at 0.00 defines the directionality of impact—to the left is negative impact, and to the right is positive impact on the model output. Here, the SHAP analysis output was with respect to the reference class which is “Low.”

A feature having black values towards the right of the vertical line at 0.00 indicates higher values of this feature contribute positively to the model output. Vice versa, a feature having black values towards the left of the vertical line at 0.00 indicates higher values of this feature contribute negatively to the model output. Similarly, a feature having grey values towards the right of the vertical line at 0.00 indicates lower values of this feature contribute positively to the model output. Vice versa, a feature having grey values towards the left of the vertical line at 0.00 indicates lower values of this feature contribute negatively to the model output.

As can be seen from FIGS. 2A and 2B, as an example, an important feature for this model to predict Low versus notLow Fprau amount was related to inositol (Inositol in g).

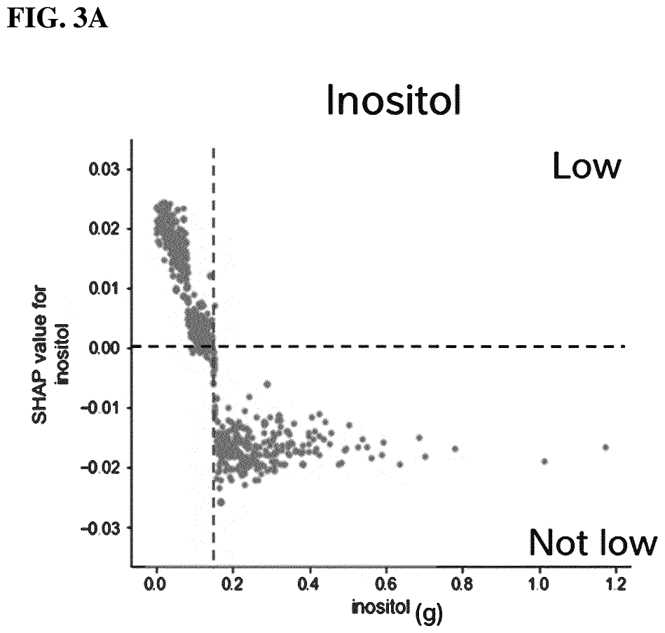

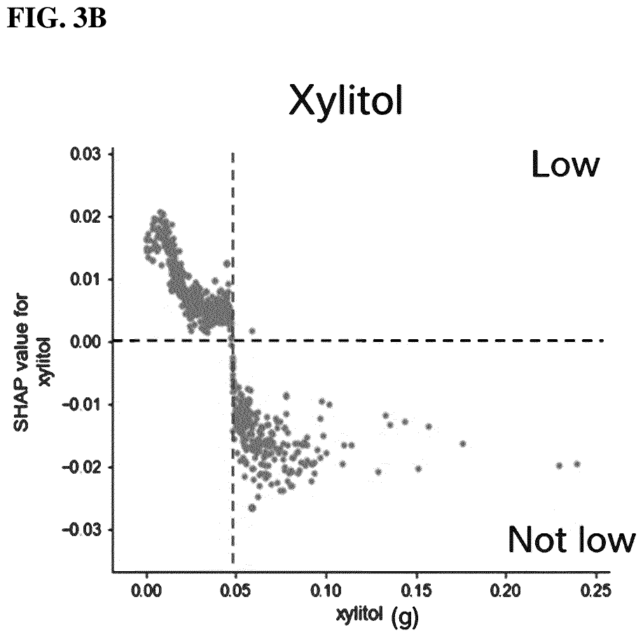

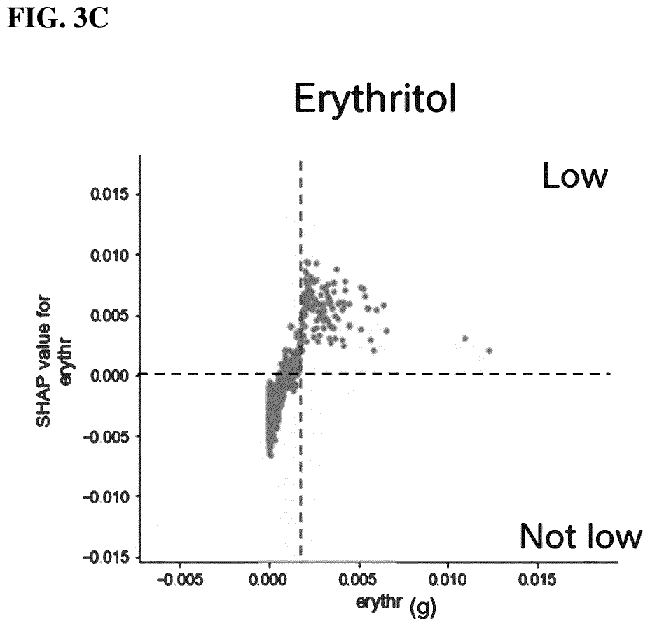

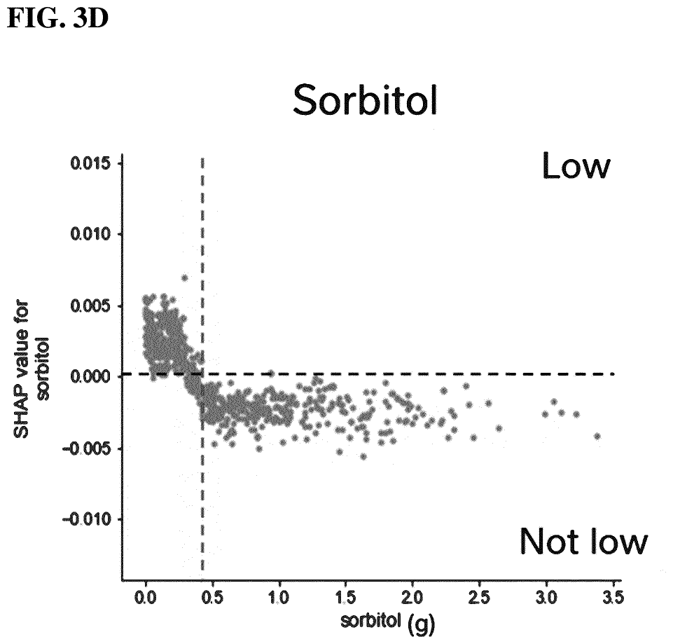

In FIGS. 3A-3D, 5A, 6A-6C, 7A and 7B, per feature, the SHAP Dependence Plot showed for each data instance/sample, the points with the feature value on the x-axis and the corresponding Shapley value on the y-axis. SHAP explained the prediction of each instance by computing the contribution of each feature to the prediction. Shapley value explanation was represented as an additive feature attribution method, as a linear model. The reference class here was “Low”, so the positive coefficients of SHAP value for the corresponding x-values of the feature indicate how much the model was affected by this feature in predicting the “Low” class.

Inositol impacted the Fprau amounts as can be seen here—it was amongst the very top features used by this model (FIGS. 2A and 2B). As shown in FIG. 3A, specific intake values of inositol had a relation with impact on model output—with low inositol intake tended to have the Fprau status to be on the lower side, while higher intake amounts of inositol tended to have the Fprau status to “notLow” class. Thus, Fprau status would benefit from more inositol intake from diet, preferably more than 0.2 g inositol per day.

FIGS. 3B-3D respectively depict the SHAP dependence plot for xylitol, erythritol and sorbitol.

### Example 5

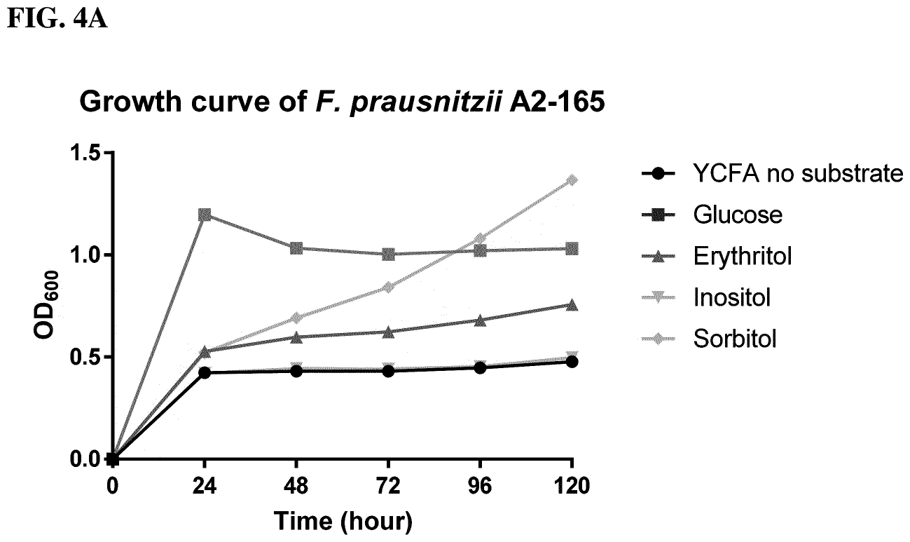

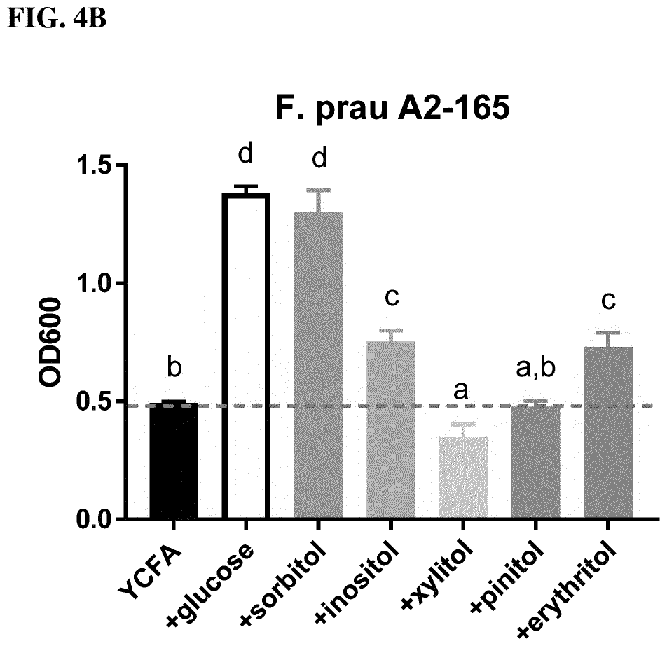

Using a model organism, the effect of inositol and sorbitol as energy substrate for F. prausnitzii was confirmed (FIGS. 4A and 4B), but not xylitol. Thus, all sugar alcohols cannot be assumed to have the same effect on F. prausnitzii.

### Example 6

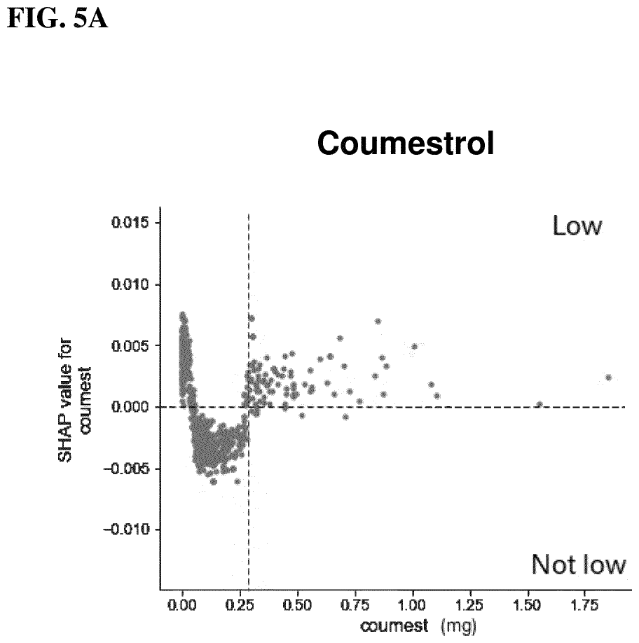

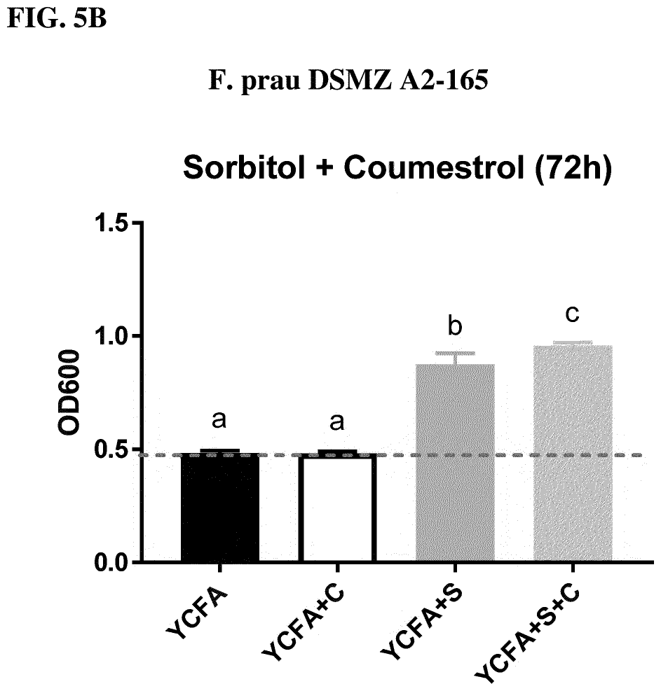

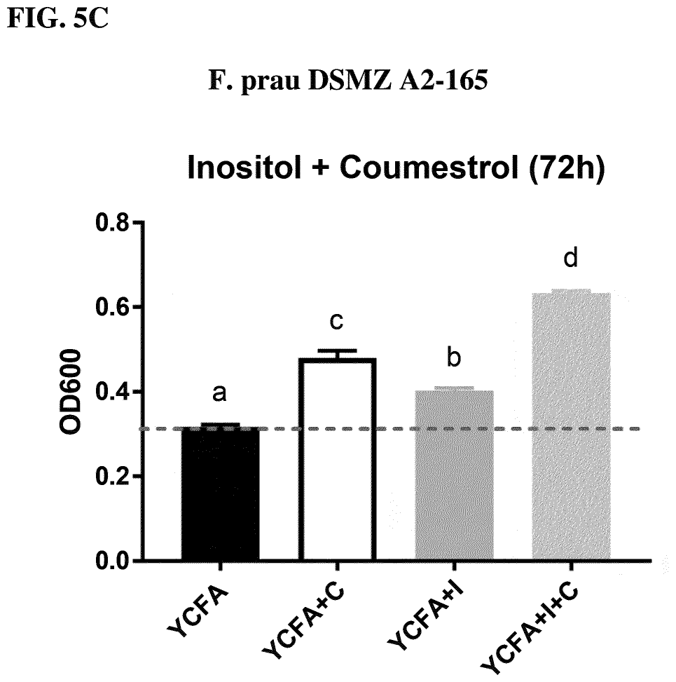

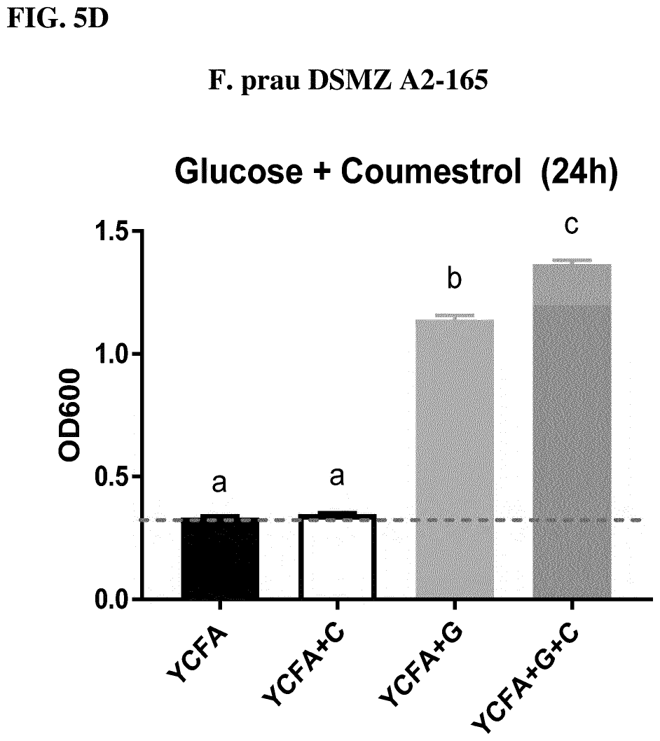

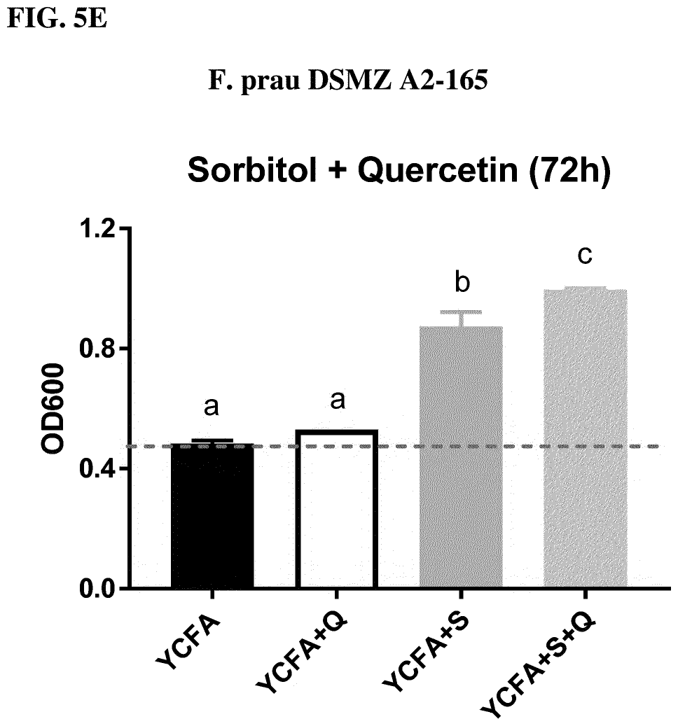

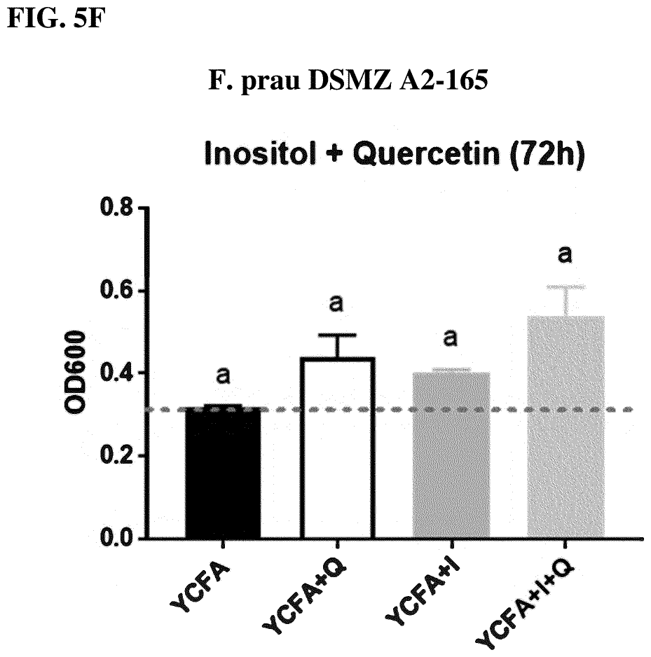

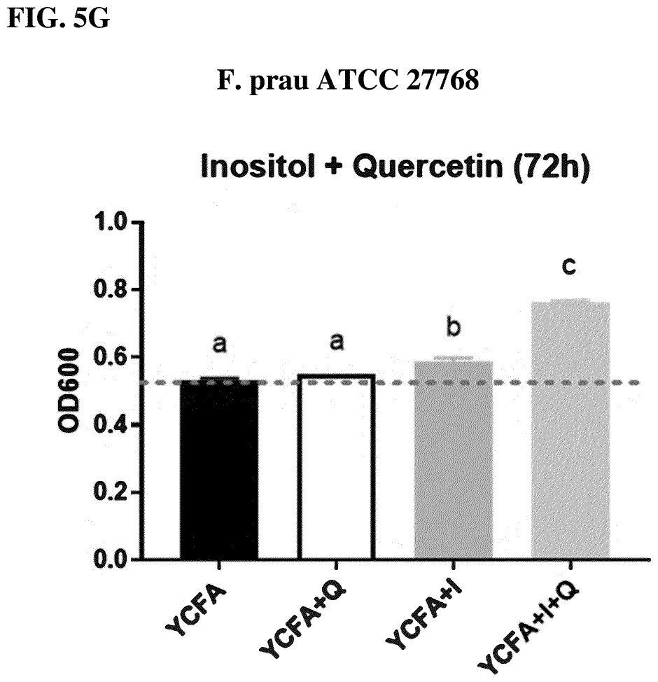

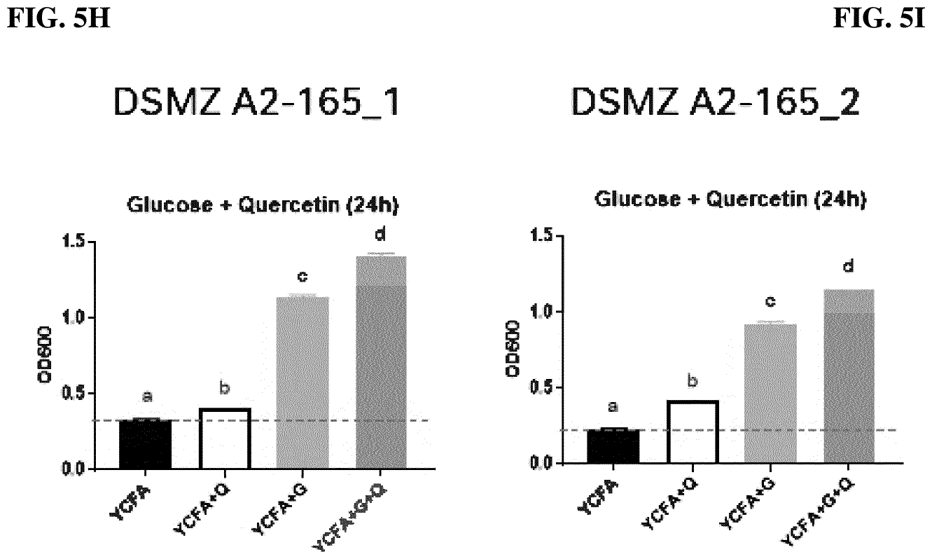

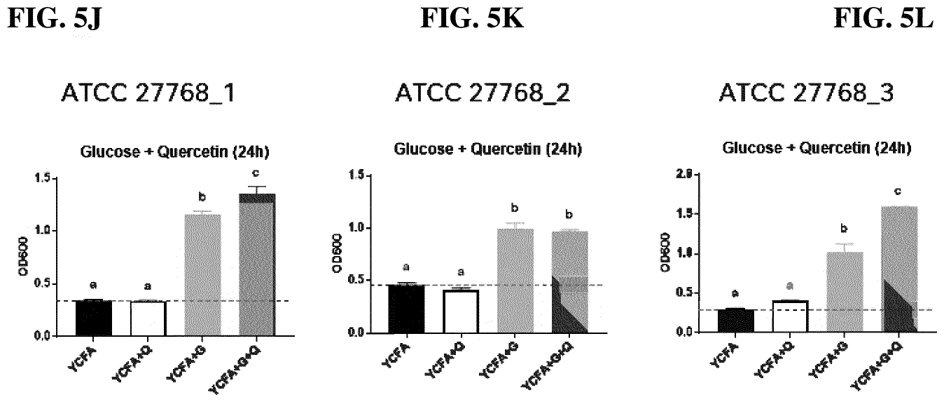

As shown in FIG. 5A, specific intake values of coumestrol had a relation with impact on model output. The results suggest a non-linear effect of coumestrol.

In vitro tests of coumestrol alone or in combinations with sorbitol and inositol are shown in FIGS. 5B and 5C. A combination of coumestrol with sorbitol or inositol enhances growth of F. prausnitzii strain DSMZ A2-165 more than each ingredient alone. As shown in FIG. 5D, a growth-promoting role in F. prausnitzii strain DSMZ A2-165 by coumestrol is also observed when combined with glucose. FIG. 5E shows growth-promoting effect in F. prausnitzii strain DSMZ A2-165 by quercetin combined with sorbitol. FIGS. 5F and 5G respectively show the growth promoting effect of quercetin with inositol for strains DSMZ A2-165 and ATCC 27768 of F. prausnitzii. FIGS. 511-5L show that growth-promoting effects of a quercetin and glucose combination is mostly positive in strains DSMZ A2-165 and ATCC 27768 of F. prausnitzii.

### Example 7

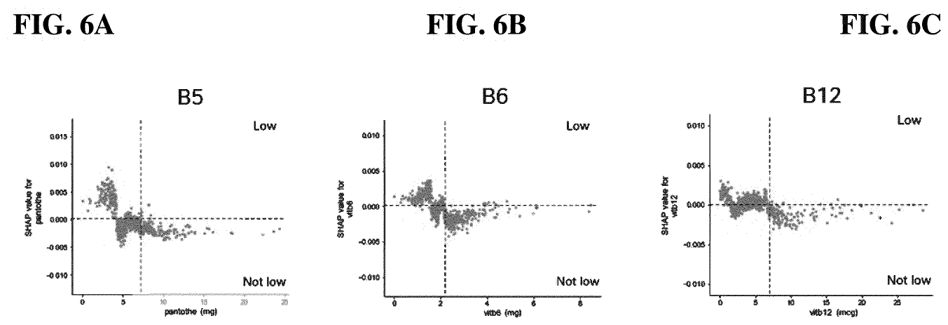

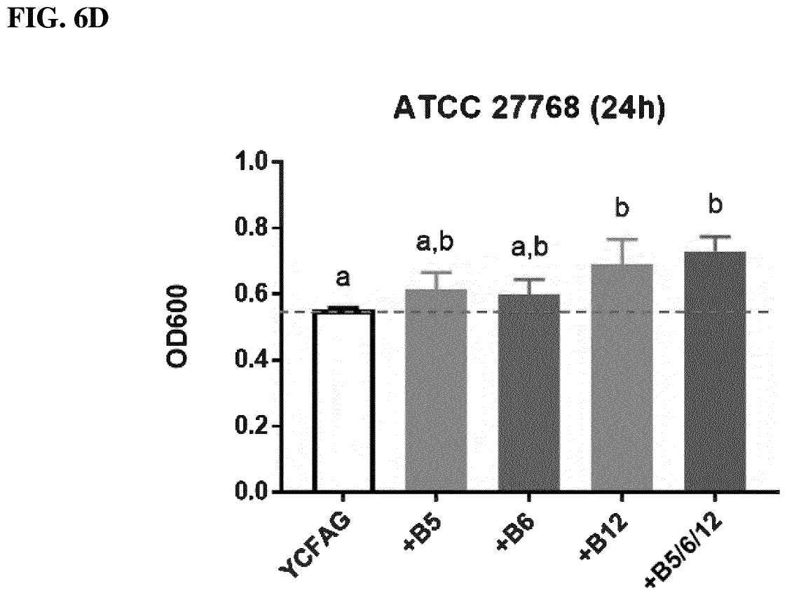

FIGS. 6A-6C show that the model identified B vitamins as nutrients contributing to growth of F. prausnitzii. FIG. 6D shows that addition of Vitamin B12 and a mix of Vitamins B5, B6 and B12 each enhance the growth of F. prausnitzii more than control when using glucose as a substrate.

### Example 8

As can be seen from FIGS. 2A and 2B, as an example, an important feature for the model to predict Low versus notLow Fprau amount was related to Vitamin A (vita_iu—Total Vitamin A Activity in IU, vita_rae—Total Vitamin A Activity Retinol Activity Equivalents in mcg, and vita_re—Total Vitamin A Activity Retinol Equivalents in mcg).

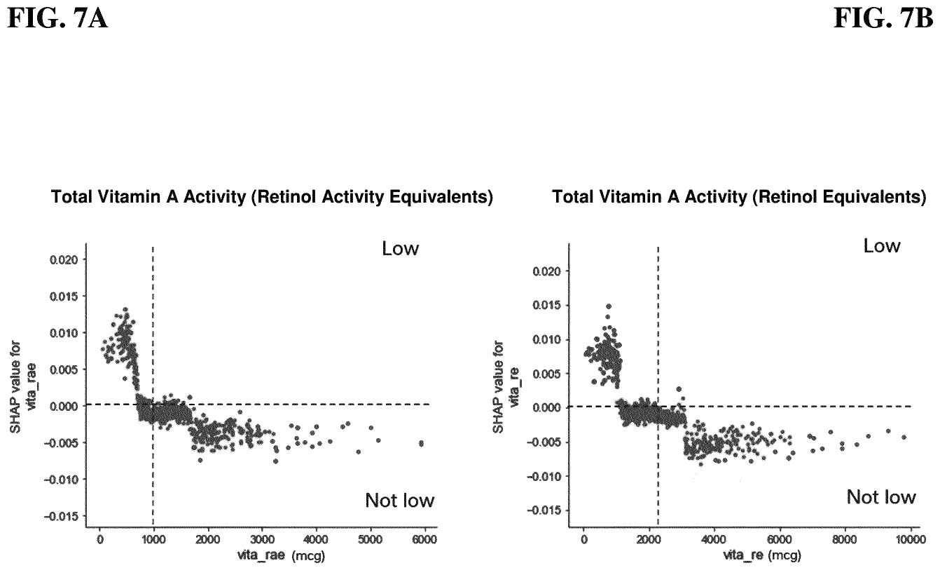

FIGS. 7A and 7B indicate increasing amounts of Vitamin A have a desired effect on Fprau amounts. This effect was captured in AGP data as vita_rae (Total Vitamin A Activity Retinol Activity Equivalents in mcg) and vita_re (Total Vitamin A Activity Retinol Equivalents in mcg). 1 IU of retinol is equivalent to approximately 0.3 micrograms (300 nanograms). As per FIGS. 7A and 7B, vita_rae>2000 mcg, and vita_re>3000 mcg had desired effects on Fprau amounts.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### REFERENCES

[1] Louis, P., Flint, H. J. (2009) Diversity, metabolism and microbial ecology of butyrate-producing bacteria from the human large intestine. FEMS Microbiol Lett. 294(1):1-8.
[2] Nicholson, J. K., Holmes, E, Kinross, J., Burcelin, R., Gibson, G. et al. (2012) The Microbial Metabolites, Short-Chain Fatty Acids, Regulate Colonic Treg Cell Homeostasis Science 336, Issue 6086, pp. 1262-1267.
[3] Smith, P. M., Howitt, M. R., Panikov, N., Michaud, M., Gallini, C. M. et al. (2013) Science 341, Issue 6145, pp. 569-573.
[4] Sokol, H, Pigneur, B., Watterlot, L., Lakhdari, O., Bermúdez-Humarin, L. G., et al. (2008) Faecalibacterium prausnitzii is an anti-inflammatory commensal bacterium identified by gut microbiota analysis of Crohn disease patients PNAS, 105 (43) 16731-16736.
[5] Wang, T., Cai, G., Qiu, Y., Fei, N., Zhang, M., et al. (2012) Structural segregation of gut microbiota between colorectal cancer patients and healthy volunteers. ISME J. 6(2):320-9.
[6] Chassard, C., Dapoigny, M., Scott, K. P., Crouzet, L., Del'homme, C., et al (2012). Functional dysbiosis within the gut microbiota of patients with constipated-irritable bowel syndrome. Aliment Pharmacol Ther. 35(7):828-38.
[7] Qin, J., Li, Y., Cai, Z., Li, S., Zhu, J., et al. (2012). A metagenome-wide association study of gut microbiota in type 2 diabetes. Nature. 490(7418):55-60.
[8] Le Chatelier, E., Nielsen, T., Qin, J., Prifti, E., Hildebrand, F., et al. (2013) MetaHIT consortium, Bork P, Wang J, Ehrlich SD, Pedersen O. Richness of human gut microbiome correlates with metabolic markers. Nature. 500(7464):541-6. doi: 10.1038/nature12506.
[9] Vrieze, A., Van Nood, E., Holleman, F., Salojarvi, J., Kootte, R. S. et al. (2012). Transfer of intestinal microbiota from lean donors increases insulin sensitivity in individuals with metabolic syndrome. Gastroenterology. 143(4):913-6.
[10] Miquel, S. et al. Current opinion in microbiology, 2013.
[11] Ferreira-Halder, C. V. et al. Clinical gastroenterology, 2017.
[12] Quévrain, E. et al. Gut, 2016.
[13] Sokol, H. et al. PNAS, 2008.
[14] Machiels, K. et al. Gut, 2014.
[15] Takahashi, K. et al. Digestion, 2016.
[16] Demirci, M. et al. Allergologia et immunopathologia, 2019.
[17] Zhao H., Xu H., Chen S., He J., Zhou Y., Nie Y., 2020. J Gastroenterol Hepatol.
[18] Machiels K. et al., Gut. 2014.
[19] Jackson M. A. et al. Genome Med. 2016.

1-9. (canceled)

10. A method to treat, prevent, reduce a severity of, and/or reduce an incidence of a pathological condition in a subject, the method comprising enhancing growth of Faecalibacterium prausnitzii in the microbiome of the subject by administering, to the subject, at least one of inositol, erythritol or sorbitol.

11. The method of claim 10, wherein the subject is selected from the group consisting of a human infant, a human child, a human adolescent, a human adult and an elderly human.

12. The method of claim 10, wherein the at least one of inositol, erythritol or sorbitol is administered in a composition further comprising at least one of a polyphenol or a vitamin.

13. The method of claim 12, wherein the polyphenol comprises at least one of coumestrol or quercetin.

14. The method of claim 12, wherein the vitamin comprises at least one of Vitamin B5, Vitamin B6, Vitamin B12, Vitamin A or Vitamin D.

15. The method of claim 10, wherein the at least one of inositol, erythritol or sorbitol is orally administered to the subject.

16. The method of claim 10, wherein the pathological condition comprises at least one of a metabolic condition selected from the group consisting of, an inflammatory condition, inflammatory bowel disease, child allergy, multiple sclerosis, aging, and frailty.

17. A method to achieve at least one of a metabolic effect, an anti-aging effect, or an anti-inflammatory effect in a subject, the method comprising enhancing growth of Faecalibacterium prausnitzii in the microbiome of the subject by administering, to the subject, at least one of inositol, erythritol or sorbitol.

18. The method of claim 17, wherein the subject is selected from the group consisting of a human infant, a human child, a human adolescent, a human adult and an elderly human.

19. The method of claim 17, wherein the at least one of inositol, erythritol or sorbitol is administered in a composition further comprising at least one of a polyphenol or a vitamin.

20. The method of claim 19, wherein the polyphenol comprises at least one of coumestrol or quercetin.

21. The method of claim 19, wherein the vitamin comprises at least one of Vitamin B5, Vitamin B6, Vitamin B12, Vitamin A or Vitamin D.

22. The method of claim 17, wherein the at least one of inositol, erythritol or sorbitol is orally administered to the subject.

23. (canceled)

24. A method of making a composition to enhance growth of Faecalibacterium prausnitzii in the microbiome of a subject to whom the composition is administered, the method comprising adding at least one of inositol, erythritol or sorbitol to at least one additional component.

25. The method of claim 24, wherein the at least one additional component comprises at least one of a polyphenol or a vitamin.

26. The method of claim 25, wherein the polyphenol comprises at least one of coumestrol or quercetin.

27. The method of claim 25, wherein the vitamin comprises at least one of Vitamin B5, Vitamin B6, Vitamin B12, Vitamin A or Vitamin D.

28. The method of claim 24, wherein the at least one additional component comprises at least one of an amino acid, a protein, a carbohydrate, or a lipid.
